(19) United States
(12) Patent Application Publication
(10) Pub. No.: US 2006/0115435 A1
(43) Pub. Date: Jun. 1, 2006

(54) Dental toothpaste, solution, tablet and system with plaque color indicator and integrated decolorizing compound

(76) Inventor: Harald Wilkens, Braunschweig (DE)

Correspondence Address: LERNER GREENBERG STEMER LLP, P O BOX 2480, HOLLYWOOD, FL, 33022-2480, US

(21) Appl. No.: 11/125,575

(22) Filed: May 9, 2005

Related U.S. Application Data
(63) Continuation of application No. PCT/DE03/03636, filed on Oct. 31, 2003.

(30) Foreign Application Priority Data
Nov. 9, 2002 (DE) 102 54 337.2

(51) Int. Cl. A61K 8/96; A61K 8/49

(52) U.S. Cl. 424/49; 424/50

(57) ABSTRACT

Thoroughly cleaning teeth daily is indispensable for keeping human teeth healthy. However, plaque is generally not sufficiently removed because it is difficult to see. Conventional plaque-dyeing agents allow plaque to be seen easily but are complicated to apply and leave traces of dyes. A toothpaste includes an integrated plaque indicator and allows plaque to be temporarily dyed. A storage container has two separate chambers, one containing toothpaste mixed with dyeing agent and the other containing toothpaste mixed with decolorizing agent. The two pastes blend when the quantity required for cleaning teeth is dispensed. Even though decolorizing begins right away, it is slow enough to obtain an intensive initial dyeing effect. The dyeing substance spreads inside the mouth during brushing and allows plaque to be easily seen and removed by the user. A decolorization rate is calculated for removing plaque within an adequate period, with all visible dye disappearing after several minutes. A self-decolorizing dyeing solution and self-decolorizing dyeing tablets for professional use are also disclosed.

### CROSS-REFERENCE TO RELATED APPLICATIONS

This application is a Continuation-In-Part, under 35 U.S.C. § 120, of copending International Application No. PCT/DE2003/003636, filed Oct. 31, 2003, which designated the United States; this application also claims the priority, under 35 U.S.C. § 119, of German Patent Application No. 102 54 337.2, filed Nov. 9, 2002; the prior applications are herewith incorporated by reference in their entirety.

### BACKGROUND OF THE INVENTION

### Field of the Invention

The invention relates to a dental toothpaste having a plaque color indicator and an integrated decolorizing compound. The invention also relates to a dental solution, a dental tablet and a dyeing system, having the plaque color indicator and integrated decolorizing compound.

Dental plaque is formed by colonies of harmful bacteria, which cannot be flushed away by simply rinsing with water. Instead, active brushing of the teeth is required to remove such adherent plaque. The application of plaque disclosing compositions for identifying plaque areas in the mouth is well known. The use of disclosing compositions motivate a person toward the removal of dental plaque by showing the presence and quantity of plaque. Commonly used disclosing agents include organic synthetic or natural dyes.

A drawback of the currently used dental compositions containing dyes for disclosing dental plaque is their prolonged staining effect. They stain the oral cavity very effectively and are difficult to remove. Some users might therefore feel uncomfortable using such disclosing dyes.

Additionally, conventional liquid staining solutions employ a relatively large amount of dye. As a result, excess staining of the entire oral cavity, as well as non-oral objects, normally results when those aqueous solutions are used.

### SUMMARY OF THE INVENTION

It is accordingly an object of the invention to provide a dental toothpaste, a dental solution, a dental tablet and a dyeing system with a plaque color indicator and an integrated decolorizing compound, which overcome the hereinafore-mentioned disadvantages of the heretofore-known products of this general type and which provide a composition for disclosing dental plaque, wherein the dye of the composition should only stain the oral cavity for a time which is sufficient for an appropriate and thorough cleaning of the same and wherein the staining should disappear after a short period of time.

The present invention overcomes these and other problems by providing a toothpaste, a dental solution and a dental tablet having an integrated plaque color indicator and an integrated decolorizing compound. In particular, the integrated decolorizing compound has at least one oxidizing agent and/or exhibits a pH≦7.

The plaque color indicator renders plaque on teeth visible to the naked eye immediately upon contact, i.e. by brushing, rinsing or chewing. The application of such an indicator normally results in an excess staining of the entire oral cavity, as well as non-oral objects. The staining disappears in a timely manner by including a decolorizing compound into the dental composition. The combination of color indicator and decolorizing compound according to the invention enables the user to clean his or her teeth thoroughly but without a long-lasting unpleasant staining of the oral cavity. This is especially useful for a substantial cleaning of the oral cavity in public places, e.g. the work place.

The oxidizing agent includes an inorganic or organic oxidizing compound or a degrading enzyme. Suitable inorganic oxidizing compounds preferably include H₂O₂, carbamide peroxide and/or sodium perborate. Tert.-butylhydroperoxid, peracetic acid and/or methyl ethyl ketone peroxide may be used as organic oxidizing compounds.

Enzymes which are able to degrade the color indicators are either oxidizing enzymes or hydrolyzing enzymes. Oxidizing enzymes such as catalases, peroxidases or hydroxylases are especially well known for their capability of oxidizing the most commonly used dyes. However, lipases and esterases used as hydrolytic enzymes are also capable of at least a partial degradation of complex ester groups containing compounds.

According to a preferred embodiment of the invention the integrated plaque color indicator shows color in alkaline solution, but is colorless in neutral or acidic solution with a pH≦7. The decoloration process is simply due to changes of the pH value. No oxidizing agents need to be added to the toothpaste, dental solution or dental tablets.

The compositions of the present invention include physiologically and pharmacologically harmless substances as color indicators which have a high affinity for adsorption by the dental plaque formations. The indicators include a member of a group of substances with heteroaromatic ring systems or chromophoric groups which are dyes or pigments. These may include phenolphthalein, erythrosine, vitamin B12, sanguinarine salts, betamine, Color Treme R-333, Color Treme R-111 and/or Patent Blue V.

The plaque color indicator and the decolorizing compound are preferably stored separately. The toothpaste according to the invention therefore includes two separate paste components. The plaque color indicator paste component is stored in a first reservoir or section and the decolorizing compound paste component is stored in a second reservoir or section. Shortly before their application, the two pastes are mixed. The storage in different reservoirs or sections circumvents a premature degradation of the color indicator by the decolorizing compound.

Another object of the invention is to provide a dental solution including an integrated plaque indicator and an integrated decolorizing compound. The ingredients of the dental solution are mainly the same as described above for the toothpaste.

Other features which are considered as characteristic for the invention are set forth in the appended claims.

Although the invention is described herein as embodied in a dental toothpaste, a dental solution, a dental tablet and a dyeing system with a plaque color indicator and an integrated decolorizing compound, it is nevertheless not intended to be limited to the details given, since various modifications and structural changes may be made therein without departing from the spirit of the invention and within the scope and range of equivalents of the claims.

The construction and method of operation of the invention, however, together with additional objects and advantages thereof will be best understood from the description of specific embodiments given below when read in connection with the following examples given as illustrations in order to point out more fully the nature of the present invention.

### EXAMPLE 1

A toothpaste including a plaque color indicator and a decolorizing compound exhibits a pH≦7. The mode of decolorization is caused by a simple color change of the indicator due to its pH-dependent color.

The toothpaste in a first reservoir or section a) contains phenolphthalein as a plaque color indicator. The pH of the reservoir or section a) is adjusted to a pH of 8.5 using a NaOH-solution. Therefore, the toothpaste in the reservoir or section a) is of reddish color. All of the other ingredients are commonly used. The pH of the toothpaste in a second reservoir or section b) is adjusted to 5 by using a concentrated H₃PO₄ solution.

Toothpaste in Reservoir or Section a:

| Ingredients | Concentration (wt %) |
|---|---|
| chalk | 35 |
| Sodium laurylsulfate | 2 |
| cellulose | 1 |
| silica | 2 |
| sorbitol | 10 |
| glycerol | 5 |
| sodium saccharinate | 0.1 |
| p-Hydroxybenzoate methylester | 0.1 |
| aminefluoride | 1 |
| water | 42.8 |
| phenolphthalein | 1 |

Toothpaste in Reservoir or Section b:

| Ingredients | Concentration (wt %) |
|---|---|
| chalk | 35 |
| Sodium laurylsulfate | 2 |
| cellulose | 1 |
| silica | 2 |
| sorbitol | 10 |
| glycerol | 5 |
| sodium saccharinate | 0.1 |
| p-Hydroxybenzoate methylester | 0.1 |
| aminefluoride | 1 |
| water | 43.8 |

### EXAMPLE 2

A toothpaste including the dye Patent Blue V as a plaque color indicator and sodium perborate as an oxidizing agent causes decolorization of the indicator.

Toothpaste in Reservoir or Section a:

| Ingredients | Concentration (wt %) |
|---|---|
| chalk | 35 |
| Sodium laurylsulfate | 2 |
| cellulose | 1 |
| silica | 2 |
| sorbitol | 10 |
| glycerol | 5 |
| sodium saccharinate | 0.1 |
| p-Hydroxybenzoate methylester | 0.1 |
| aminefluoride | 1 |
| water | 41.8 |
| Patent Blue V | 2 |

Toothpaste in Reservoir or Section b:

| Ingredients | Concentration (wt %) |
|---|---|
| chalk | 25 |
| Sodium laurylsulfate | 2 |
| cellulose | 1 |
| silica | 2 |
| sorbitol | 10 |
| glycerol | 5 |
| sodium saccharinate | 0.1 |
| p-Hydroxybenzoate methylester | 0.1 |
| aminefluoride | 1 |
| water | 33.8 |
| sodium perborate | 20 |

### EXAMPLE 3

The dye Patent Blue V is used as a plaque color indicator and a catalase as an oxidizing enzyme causing oxidation and degradation of the indicator.

Toothpaste in Reservoir or Section a:

| Ingredients | Concentration (wt %) |
|---|---|
| chalk | 35 |
| Sodium laurylsulfate | 2 |
| cellulose | 1 |
| silica | 2 |
| sorbitol | 10 |
| glycerol | 5 |
| sodium saccharinate | 0.1 |
| p-Hydroxybenzoate methylester | 0.1 |
| aminefluoride | 1 |
| water | 41.8 |
| Patent Blue V | 2 |

Toothpaste in Reservoir or Section b:

| Ingredients | Concentration (wt %) |
|---|---|
| chalk | 30 |
| Sodium laurylsulfate | 2 |
| cellulose | 1 |
| silica | 2 |
| sorbitol | 10 |
| glycerol | 5 |
| sodium saccharinate | 0.1 |
| p-Hydroxybenzoate methylester | 0.1 |
| aminefluoride | 1 |
| water | 38.8 |
| catalase | 10 |

It is understood that the foregoing detailed description is given merely by way of illustration and that variations may be made therein without departing from the spirit from the invention.

I claim:

1. A toothpaste, comprising:
an integrated plaque dye indicator, said plaque dye indicator so clearly visibly dyeing dental plaque as to be definitely discernible to a user and effectively removable; and
an integrated decolorizing agent, said integrated decolorizing agent causing residues of said plaque dye indicator remaining in an oral cavity to be of neutral color again after a short time.

2. The toothpaste according to claim 1, wherein said integrated plaque dye indicator is a dye, and said dye is nontoxic, has a good diffusibility in dental plaque, is matched to said integrated decolorizing agent so as to only appear colored in alkaline solution, and is bleachable or oxidizable and enzymatically decomposable.

3. The toothpaste according to claim 1, wherein said integrated plaque dye indicator is a dye, and said integrated decolorizing agent is nontoxic, is readily mixable with said dye, is slightly acidic or neutral depending on said dye being used, and contains a bleaching agent and an enzyme decomposing said dye.

4. A plaque dyeing system, comprising:
a two-chamber container having a first reservoir storing an integrated plaque dye indicator, and a second reservoir storing an integrated decolorizing agent as a solution separately from said plaque dye indicator;
said plaque dye indicator being a nontoxic dye having a good diffusibility in dental plaque, being matched to said decolorizing agent so as to only appear colored in alkaline solution, and being bleachable or oxidizable and enzymatically decomposable;
said decolorizing agent being nontoxic, readily mixable with said dye, slightly acidic or neutral depending on said dye being used, and containing a bleaching agent and an enzyme decomposing said dye; and
said dye and said decolorizing agent to be mixed immediately before use to form a mixture having a temporary dyeing action briefly showing the dental plaque clearly in color and causing dye residues remaining in an oral cavity to be neutral in color again after a short time.

5. A plaque dyeing tablet, comprising: at least one bite-through capsule having an integrated plaque dye indicator and an integrated decolorizing agent;
said plaque dye indicator being a nontoxic dye having a good diffusibility in dental plaque, being matched to said integrated decolorizing agent so as to only appear colored in alkaline solution, and being bleachable or oxidizable and enzymatically decomposable;
said integrated decolorizing agent being nontoxic, readily mixable with said dye, slightly acidic or neutral depending on said dye being used, and containing a bleaching agent and an enzyme decomposing said dye; and
said dye and said decolorizing agent forming a mixture having a temporary dyeing action briefly showing the dental plaque clearly in color and causing dye residues remaining in an oral cavity to be neutral in color again after a short time.

6. A toothpaste, comprising an integrated plaque color indicator and an integrated decolorizing compound, said decolorizing compound having at least one oxidizing agent and/or a pH value at most equal to 7.

7. The toothpaste according to claim 6, wherein said oxidizing agent is selected from the group consisting of an inorganic oxidizing compound, an organic oxidizing compound and a degrading enzyme.

8. The toothpaste according to claim 7, wherein said inorganic oxidizing compound is selected from the group consisting of H₂O₂, carbamide peroxide and sodium perborate.

9. The toothpaste according to claim 7, wherein said organic oxidizing compound is selected from the group consisting of tert.-butylhydroperoxid, peracetic acid and methyl ethyl ketone peroxide.

10. The toothpaste according to claim 7, wherein said degrading enzyme is at least one enzyme selected from the group consisting of an oxidizing enzyme and a hydrolyzing enzyme.

11. The toothpaste according to claim 10, wherein said oxidizing enzyme is selected from the group consisting of a catalase, a peroxidase, and a hydroxylase.

12. The toothpaste according to claim 10, wherein said hydrolyzing enzyme is selected from the group consisting of a lipase and an esterase.

13. The toothpaste according to claim 6, wherein said integrated plaque color indicator is colored in alkaline solution and is colorless in neutral or acidic solution.

14. The toothpaste according to claim 6, wherein said integrated plaque color indicator includes at least one compound with a heteroaromatic ring system or a chromophoric group.

15. The toothpaste according to claim 14, wherein said integrated plaque color indicator includes at least one of phenolphthalein, erythrosine, vitamin B12, sanguinarine salts, betamine, Color Treme R-333, Color Treme R-111 or Patent Blue V.

16. The toothpaste according to claim 6, wherein said integrated plaque color indicator and said integrated decolorizing compound are stored in separate respective paste components.

17. The toothpaste according to claim 16, which further comprises a toothpaste storage device having first and second reservoirs, said paste component having said integrated plaque color indicator being stored in said first reservoir and said paste component having said integrated decolorizing compound being stored in said second reservoir.

18. The toothpaste according to claim 16, wherein said paste component having said integrated plaque color indicator and said paste component having said integrated decolorizing compound are mixed shortly before application.

19. A dental solution, comprising an integrated plaque color indicator and an integrated decolorizing compound, said decolorizing compound having at least one oxidizing agent and/or a pH value at most equal to 7.

20. A dental tablet, comprising an integrated plaque color indicator and an integrated decolorizing compound, said decolorizing compound having at least one oxidizing agent and/or a pH value at most equal to 7.
